(19) United States
(12) Plant Patent Application Publication
(10) Pub. No.: US 2013/0061360 P1
(43) Pub. Date: Mar. 7, 2013

(54) Buddleia plant named 'Hinebud1'

(50) ;

(71) Applicant: Peter C. Podaras, Davis, CA (US)

(72) Inventor: Peter C. Podaras, Davis, CA (US)

(73) Assignee: Hines Growers LLC, Laguna Hills, CA (US)

(21) Appl. No.: 13/199,738

(22) Filed: Sep. 7, 2011

(51) Int. Cl. A01H 5/00

(52) U.S. Cl. PLT/242

(57) ABSTRACT

A Buddleia plant particularly distinguished by a low-growing and spreading growth habit, large, pendulous inflorescence and suitable for use in hanging baskets, containers, and beddings, is disclosed.

### GENUS AND SPECIES

Buddleia hybrid

### VARIETY DENOMINATION

‘Hinebud1’

### BACKGROUND OF THE NEW PLANT

The present invention comprises a new and distinct variety of Buddleia, botanically known as Buddleia hybrid, and hereinafter referred to by the variety name ‘Hinebud1’. The new variety originated from a cross-pollination conducted in Winters, Calif. in July 2010 between the female Buddleia parent, an un-named seedling (unpatented) and the male Buddleia parent, ‘Podaras #12’ U.S. Plant Pat. No. 22,098). The female parent is a complex hybrid of Buddleia davidii×Buddleia alternifolia×Buddleia caryopterifolia×Buddleia lindleyana×Buddleia crispa. The male parent is a hybrid of Buddleia davidii×Buddleia alternifolia)×Buddleia davidii. A single plant selection was subsequently chosen for further evaluation and asexual propagation.

The new plant was first asexually propagated on Sep. 15, 2010 in Winters, Calif. and has been asexually reproduced by cuttings for three generations in Winters, Calif. The present invention has been found to retain its distinctive characteristics through successive asexual propagations by softwood cuttings.

‘Hinebud1’ has not been made publicly available or sold more than one year prior to the filing of this application.

### SUMMARY OF THE INVENTION

The following are the most outstanding and distinguishing characteristics of the new variety when grown under normal horticultural practices in Winters, Calif.

1. A low-growing and spreading growth habit;

2. Large, pendulous inflorescence; and

3. For use in hanging baskets, containers and beddings.

### DESCRIPTION OF THE PHOTOGRAPHS

This new Buddleia plant is illustrated by the accompanying photographs which show the overall plant habit, foliage and inflorescence of the plant. The colors are as true as can be reasonably obtained by conventional photographic procedures. The photographs are of a one-year old plant grown in a greenhouse and outdoors in Winters, Calif. in August 2011.

FIG. 1 shows the whole plant, including the habit, foliage and inflorescence.

FIG. 2 shows a close-up of the inflorescence and buds.

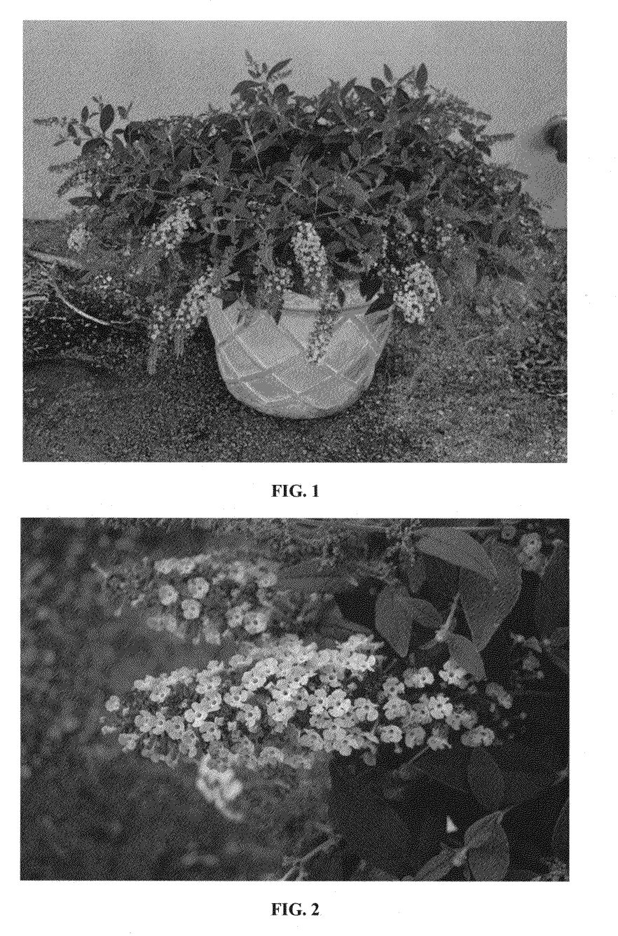

### DESCRIPTION OF THE NEW VARIETY

The following detailed descriptions set forth the distinctive characteristics of ‘Hinebud1’. The data which defines these characteristics were collected from asexual reproductions carried out in Winters, Calif. The plant history was taken in August 2011 on one-year old plants grown in a greenhouse and outdoors in Winters, Calif. Color references were taken under natural light and are to the R.H.S. Colour Chart of The Royal Horticultural Society of London (R.H.S.), fifth edition (2007). Texture descriptions were taken using a dissecting microscope.

### DESCRIPTION OF THE NEW PLANT

Classification:
   Family.—Scrophulariaceae.
   Botanical name.—Buddleia hybrid.
   Common name.—Butterfly bush.
   Denomination.—‘Hinebud1’.
Plant:
   Form.—Shrub.
   Habit.—Low growing and spreading.
   Height (measured from the top of the soil).—38.0 cm.
   Width (horizontal plant diameter).—65.0 cm.
   Time to produce a finished plant.—10 to 12 weeks from a cutting.
   Outdoor plant performance.—Excellent and prefers full sun.
   Time to initiate and develop roots.—10 to 14 days.
   Root description and habit.—White, fibrous, dense and branching.
   Propagation type.—Softwood cuttings.
Lateral branches:
   Quantity per plant.—Approximately 30.
   Length.—30.0 cm to 45.0 cm.
   Internode length.—5.0 cm.
   Diameter (from midpoint).—0.35 cm.
   Color.—RHS 148B for mature stems.
   Strength.—Flexible.
   Texture.—Slightly pubescent.
   Anthocyanin.—RHS N186B.
Leaves:
   Quantity of leaves per branch.—8 to 10.
   Arrangement.—Opposite.
   Length.—4.5 cm to 9.5 cm, with the average being 8.5 cm.
   Width.—Up to 3.2 cm.
   Shape.—Lanceolate.
   Apex.—Broadly acute.
   Base.—Acute.
   Margin.—Serrate.
   Color, immature.—Upper surface: RHS 139A Lower surface: RHS 191B.
   Color, mature.—Upper surface: RHS 137A Lower surface: RHS 191B.
   Texture.—Upper surface: Mostly resinous with some oppressed, stellate trichomes Lower surface: Resinous and densely covered with oppressed overlapping, stellate hairs.
   Venation type.—Reticulate.
   Venation color.—Upper surface: RHS 137A Lower surface: RHS 191A.
   Petiole.—Color: RHS 146B Length: 0.5 cm Diameter: 0.2 cm Texture: Resinous with many stellate trichomes.
Inflorescence:
   Height.—15.0 cm to 18.0 cm.
   Width.—4.5 cm.
   Bud.—Color: RHS 191C Shape: Conical Height: 1.5 cm Diameter: 0.8 cm.
   Flower.—Type and habit: Single and salverform shaped flowers arranged in compound terminal panicles; flowers face upright and outward; panicles are roughly conical in shape Fragrance: Sweetly scented Flowering season: Early summer to fall Lastingness of inflorescence on the plant: About one week Depth: 1.3 cm to 1.5 cm Diameter: 1.1 cm Quantity per inflorescence: 180 to 250 Petals: Arrangement and quantity per flower: Four arranged in a single whorl with the petals fused into a narrow tube Length: 0.4 cm Width: 0.5 cm Apex: Rounded and emarginate Base: Fused Margin: Crenate Texture: Upper surface: Very papillate Lower surface: Papillate with raised veining and scattered stellate trichomes Color, when opening: Upper surface: RHS N82B Lower surface: RHS N82B Color, when open: Upper surface: RHS N82C and fading to RHS 97B Lower surface: RHS N82C and fading to RHS 97B Tube: Length: 1.0 cm Width: 0.2 cm Color: Inside: RHS 26A Outside: RHS N77B Texture: Inside: Papillate with some resinous bodies and upward facing strigose hairs Outside: Very papillate with scattered glandular bodies Calyx: Arrangement and quantity: Four sepals arranged in a single whorl and fused towards the base Shape: Tubular, fitting snugly over the corolla tube Sepals: Length: 0.4 cm Width: 0.2 cm Shape: Ovate Apex: Broadly acute Base: Fused with other sepals Margin: Smooth (entire) Texture: Upper surface: Muricate Lower surface: Rough and uneven, stellate trichomes with some resinous bodies Color: Upper surface: RHS 139B Lower surface: RHS 138A Peduncle: Length: 2.0 cm to 4.0 cm Diameter: 0.25 cm Strength: Flexible Color: RHS N199B on parts exposed to the sun; RHS 146 on parts not exposed to the sun Texture: Densely covered with oppressed stellate trichomes Pedicel: Length: 0.1 cm to 0.3 cm Diameter: 0.1 cm Color: RHS 146C Texture: Covered with oppressed, stellate trichomes.
Reproductive organs:
   Stamens.—Quantity per flower: 4 Filament: Completely fused to inside of corolla tube; cannot determine length or color Anther: Length: 0.15 cm Color: RHS 161B Pollen amount: Moderate Pollen color: RHS 155D.
   Pistil.—Quantity per flower: 1 Length: 0.2 cm Stigma: Shape: Spatulate with minute cleft at very top Color: RHS 141B Style: Length: 0.15 cm Color: RHS 154C Ovary: Color: RHS 139C.
Fruit and seed set: Sets seed in presence of other Buddleia plants; seed color is RHS 159B and shape is long and narrow, about 0.025 cm by 0.01 cm.
Disease and insect resistance: Susceptible to spider mites.

### COMPARISON WITH PARENTAL AND COMMERCIAL VARIETY

‘Hinebud1’ differs from the un-named female Buddleia parent (unpatented) in that ‘Hinebud1’ is more compact and lower growing.

‘Hinebud1’ differs from the male Buddleia parent ‘Podaras #12’ (U.S. Plant Pat. No. 22,098) in that ‘Hinebud1’ is lower growing and has a wider growth habit.

‘Hinebud1’ differs from the commercial comparison ‘Black Knight’ (unpatented) in that ‘Hinebud1’ has a very spreading and low growing habit, while ‘Black Knight’ has a dense and upright growth habit. Additionally, ‘Hinebud1’ has a height of less than 2 feet (60.96 cm), while ‘Black Knight’ has a height of approximately 5 feet to 6 feet (152.4 cm to 183 cm).

I claim:

1. A new and distinct variety of Buddleia plant as shown and described herein.
